SEQUENCE LISTING (5 sequences)

SEQ ID NO: 1 - DNA, 4892 bp, Escherichia coli
  terminator (73)..(100): T2
gttaacgctagcatggatctcgggccccaaataatgattttattttgactgatagtgacctgttcgttgcaacaaattgatgagcaatgcttttttataatgccaactttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgcctgcaggtcgactctagaggatccccgggtaccgagctcgaattccgttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcacggaattcgagctcggtacccggggatcctctagagtcgacctgcaggcatgttctttcctgcgttatcccctgattctgtggataaccgtattaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgc

SEQ ID NO: 2 - DNA, 12 bp, Artificial Sequence
  Primer
ggaggctcttca

SEQ ID NO: 3 - DNA, 46 bp, Artificial Sequence
  Primer
ggggacaagtttgtacaaaaaagcaggcttaggaggctcttcaatg

SEQ ID NO: 4 - DNA, 12 bp, Artificial Sequence
  Primer
agctgggttcta

SEQ ID NO: 5 - DNA, 33 bp, Artificial Sequence
  Primer
ggggaccactttgtacaagaaagctgggttcta

(19) United States
(12) Patent Application Publication
(10) Pub. No.: US 2003/0219902 A1
(43) Pub. Date: Nov. 27, 2003

(54) Methods and vectors for facilitating site-specific recombination

(75) Inventors: Michael L. Kahn, Pullman, WA (US); Brent L. House, Pullman, WA (US); Michael W. Mortimer, Moscow, ID (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Washington State University Research Foundation

(21) Appl. No.: 10/357,268

(22) Filed: Jan. 31, 2003

Related U.S. Application Data
(60) Provisional application No. 60/354,063, filed on Jan. 31, 2002.

(51) Int. Cl. C12Q 1/68; C12N 15/74; C12N 1/21

(52) U.S. Cl. 435/488; 435/252.33; 435/6

(57) ABSTRACT

In one aspect the invention provides methods for moving an insert nucleic acid molecule between vectors using site-specific recombination in vivo. In another aspect, the invention provides methods for the functional analysis of a genome using site-specific recombination in vivo. Another aspect of the invention provides methods for deleting a target genomic region by intra-molecular site-specific recombination. Further aspects provide vectors and kits for use in the methods of the invention.

### CROSS-REFERENCE TO RELATED APPLICATION

This application claims the benefit of U.S. Provisional Application No. 60/354,063, filed Jan. 31, 2002, under 35 U.S.C. § 119.

### GOVERNMENT RIGHTS

This invention was made with government support under DE-FG03-99ER20225 awarded by the Department of Energy. The government has certain rights in the invention.

### FIELD OF THE INVENTION

The present invention relates to methods and vectors for using site-specific recombination in vivo to move insert nucleic acid molecules between vectors and to analyze the function of a genome, for example, by creating genomic deletions.

### BACKGROUND OF THE INVENTION

Various genetic and biochemical approaches have been used to study the biology of an organism, including array construction, reporter gene fusions, mutagenesis, protein production and characterization. These approaches generally require subcloning of DNA molecules of interest into specialized vectors for analysis. Traditional subcloning methods, using restriction enzymes and ligase, are time-consuming, expensive, and impractical for the systematic analysis of genomes. Accordingly, there is a need for efficient and economical methods of subcloning, especially for large-scale functional analyses of genomes.

Large-scale genomic sequencing has led to the identification of numerous predicted coding sequences with no known function. Targeted deletion of these predicted coding sequences is an effective approach to discover the function of such predicted coding sequences. Moreover, targeted deletion is a useful method to remove deleterious or undesirable sequences from a genome. Therefore, there is a need for methods of making targeted genomic deletions.

### SUMMARY OF THE INVENTION

In accordance with the foregoing, in one aspect the invention provides methods for moving an insert nucleic acid molecule between vectors, comprising transferring an insert nucleic acid molecule from a first vector to a second vector using site-specific recombination in vivo. In some embodiments, the methods comprise combining two or more cells, wherein each cell comprises at least one of: (a) a first vector comprising a transfer origin and an insert nucleic acid molecule, wherein the insert nucleic acid molecule is flanked by a first recombination site and by a second recombination site; (b) a second vector comprising a transfer origin and a first recombination site partner and a second recombination site partner; (c) one or more recombination proteins, which mediate recombination between the first recombination site and the first recombination site partner, and between the second recombination site and the second recombination site partner, respectively; and (d) one or more plasmid transfer factors, which provide inter-cellular transfer of vectors comprising the transfer origin; and wherein the cells are combined under conditions effective to promote conjugation and recombination. In some embodiments, the methods comprise combining a first cell comprising the first vector comprising the insert nucleic acid molecule and a second cell comprising the second vector.

In some embodiments, the methods comprise combining: (a) a first cell comprising a first vector comprising a transfer origin and an insert nucleic acid molecule, wherein the insert nucleic acid molecule is flanked by a first recombination site and by a second recombination site; (b) a second cell comprising a second vector comprising a transfer origin and a first recombination site partner and a second recombination site partner; (c) a third cell comprising one or more recombination proteins, which mediate recombination between the first recombination site and the first recombination site partner, and between the second recombination site and the second recombination site partner, respectively; and (d) a fourth cell comprising one or more plasmid transfer factors, which mediate inter-cellular transfer of the first and second vectors; wherein the cells are combined under conditions effective to promote conjugation and recombination. Some embodiments further comprise combining a fifth cell that allows for selection of the second vector comprising the insert nucleic acid molecule.

In some embodiments, the site-specific recombination system comprises the integrase/att system from bacteriophage lambda. In some embodiments, the transfer origins in the first and second vectors comprise oriT sequences from plasmid RK2 or ColE1.

In a second aspect, the invention provides methods and vectors for analyzing a genome using site-specific recombination in vivo. In some embodiments, the methods comprise (a) providing a first vector comprising a transfer origin and an insert nucleic acid coding molecule flanked by a first recombination site and by a second recombination site, wherein the insert nucleic acid molecule comprises a sequence from a genomic sequence in a first organism; (b) transferring the insert nucleic acid molecule within the first vector into a second vector comprising a transfer origin and a first recombination site partner and a second recombination site partner by site-specific recombination in a second organism; (c) transferring the second vector from the second organism into the first organism by conjugation; and (d) analyzing the function of the genomic region in the first prokaryotic organism. Typically, the first and second organisms are different prokaryotic organisms. In some embodiments, the first organism is Sinorhizobium meliloti.

In a further aspect, the invention provides methods for deleting a target region in a prokaryotic genome by intra-molecular site-specific recombination. In some embodiments, the methods comprise: (a) introducing a first recombination site into a first genomic region by homologous recombination, wherein the first genomic region is adjacent to a first end of a target genomic region; (b) introducing a second recombination site into a second genomic region by homologous recombination, wherein the second genomic region is adjacent to a second end of the target genomic region; and (c) deleting the target genomic region by providing one or more recombination proteins to catalyze site-specific recombination between the first and second recombination sites.

In some embodiments, step (a) comprises introducing a first vector comprising a first DNA sequence and a first recombination site into a first genomic region by homologous recombination, wherein the first genomic region is adjacent to a first end of a target genomic region and the first DNA sequence is homologous to the first genomic region; and step (b) comprises introducing a second vector comprising a second DNA sequence and a second recombination site into a second genomic region by homologous recombination, wherein the second genomic region is adjacent to a second end of a target genomic region, the second DNA sequence is homologous to the second genomic region, and the second recombination site has the same orientation as the first recombination site. In some embodiments, step (a) comprises. homologously recombining a first vector comprising a first DNA sequence between a first and a second recombination site into a first genomic region, wherein the first genomic region is adjacent to a first end of a target genomic region, the first DNA sequence is homologous to the first genomic region, and the first and second recombination sites have different orientations; and step (b) comprises homologously recombining a second vector comprising a second DNA sequence between a third and fourth recombination site into a second genomic region, wherein the second genomic region is adjacent to a second end of the target genomic region, the second DNA sequence is homologous to the second genomic region, the third recombination site has the same orientation as the second orientation site, and the fourth recombination site has the same orientation as the first site-specific recombination site.

In some embodiments, the recombination sites comprise FRT sequences and the one or more recombination proteins comprise a Flp recombinase. In some embodiments, the prokaryotic genome is the S. meliloti genome

In another aspect, the present invention provides an integrated and extendable set of site-specific recombination vectors that can be used for functional analyses of genomes. In some embodiments, the invention provides vectors, comprising a transfer origin for conjugation and a selectable marker flanked by a first recombination site and a second recombination site. In some embodiments, the first and second recombination sites comprise att sites. The selectable marker may be a toxic gene, such as ccdB. In some embodiments, the transfer origin comprises the oriT sequence from RK2 or ColEI. In some embodiments, the vector comprises the sequence provided in SEQ ID NO:1. The vectors of the invention may be used for the functional analysis of a bacterial genome, such as the genome of Sinorhizobium meliloti.

In another aspect, the invention provides site-specific recombination kits for the systematic functional analysis of a genome. In some embodiments, the kits comprise one or more vectors comprising a transfer origin for conjugation and a selectable marker flanked by a first recombination site and a second recombination site, and instructions for moving one or more insert nucleic acid molecules from a first vector into a second vector using site-specific recombination in vivo. The kits may further comprise cells, for example cells comprising coding sequences for one or more recombination proteins, cells comprising coding sequences for one or more transfer factors, and/or cells that allow for selection of recombinant second vectors comprising the insert nucleic acid molecule(s).

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B illustrate the methods of cloning PCR DNA and transferring it to destination vectors. The oriT sequence from plasmid RK2 was inserted into pDONR201, to give the mobilizable donor vector pMK2010. The desirable features of this plasmid are its small size, which improves transformation frequency, a selectable marker (kan) that can be used for both positive and negative selection, and ccdB, which can be used to eliminate the unrecombined plasmid. Sequences flanking the site of insertion are designed to minimize potential gene expression, to protect against potentially toxic effects of inappropriately expressed proteins. Cloning of PCR DNA into pMK2010 gives an attL entry vector, which can recombine with either an expression vector (FIG. 1A) or elimination vector (FIG. 1B) to give the plasmids used to carry out the useful manipulations shown in FIG. 4. P_{T7}=T7 promoter; Pᵢ=inducible promoter; hyg=hygromycin resistance; kan=kanamycin resistance; tet=tetracycline resistance. A similar integrase-mediated recombination transfers the cloned DNA into a replicating vector for expression, complementation, or regulation studies.

FIG. 2 shows the flow of work and identification of useful products. Numerals refer to stages in the construction shown in FIG. 4.

FIG. 3 shows the design of primers. Primers have a 20 base overlap with target sequences at the 3′ and 5′ end of an ORF and 12 bp overlap with attB. Stop codons are changed to UAG, if this is not the natural stop codon. Primer 1=first forward primer, primer 2=second forward primer, primer 3=first reverse primer, and primer 4=second reverse primer, as described in EXAMPLE 1.

FIG. 4 shows the use of the expression and elimination vectors. The box (lower right) shows the final situation for various possible orientations of genes A and C.

### DETAILED DESCRIPTION OF THE PREFEPRED EMBODIMENT

Unless specifically defined herein, all terms used herein have the same meaning as they would to one of ordinary skill in the art.

The invention provides methods and vectors for using site-specific recombination in vivo for moving insert nucleic acid molecules between vectors and for analyzing the function of a genome, for example by making targeted genomic deletions, reporter fusions or gene expression constructs. Site-specific recombinases are proteins that are present in many organisms (e.g., viruses and bacteria) and have been characterized to have both endonuclease and ligase properties. These recombinases (along with associated proteins in some cases) recognize specific sequences of bases in DNA and exchange the DNA segments flanking those segments. The recombinases and associated proteins are collectively referred to as “recombination proteins” (see, e.g., Landy (1993) Curr. Op. Biotechnol. 3:699-707).

Numerous recombination systems from various organisms have been described (see, e.g., Hoess et al. (1986) Nucl. Acids Res. 14(6):2287; Abremski et al. (1986) J. Biol. Chem. 261(1):391; Campbell (1992) J. Bacteriol. 174(23):7495; Qian et al. (1992) J. Biol. Chem. 267(11):7794; Araki et al. (1992) J. Mol. Biol. 225(1):25; Maeser & Kahnmann (1991) Mol. Gen. Genet. 230:170-176); Esposito et al. (1997) Nucl. Acids Res. 25(18):3605). Many of these belong to the integrase family of recombinases (Argos et al. (1986) EMBO J. 5:433-440). Perhaps the best studied of these are the integrase/att system from bacteriophage lambda (Landy (1993) Curr. Op. Biotechnol. 3:699-707), the Cre/loxP system from bacteriophage P1 (Hoess & Abremski (1990) In Nucleic Acids and Molecular Biology 4:90-109, Eckstein & Lilley, eds., Springer-Verlag, Berlin-Heidelberg), and the Flp/FRT system from the Saccharomyces cerevisiae 2μ circle plasmid (Broach et al. (1982) Cell 29:227-234).

The major advantage of using recombinase-assisted cloning is that, unlike cut and paste technology with restriction enzymes and DNA ligase, recombinases catalyze a concerted recombination. Their very long sequence specificity allows them to catalyze precise manipulations with large DNA molecules that would be difficult with restriction enzyme techniques.

The prototypical site-specific recombination reaction is used to integrate the lysogenic bacteriophage, lambda, into the E. coli chromosome. Two DNA sequences are involved, attP, the 243 bp phage attachment sequence, and attB, the 25 bp bacterial site of attachment (Mizuuchi & Mizuuchi (1980) Proc. Natl. Acad. Sci. U.S.A. 77:3220-4; Mizuuchi & Mizuuchi (1985) Nucleic Acids Res. 13:1193-208). Recombination catalyzed by the integrase protein, Int, occurs within a 15 bp “core” region of identity between attP and attB. Recognition of these sequences by integrase primarily involves the “arm” sequences of attP, which extend about 150 bases to the left and 100 bases to the right of the core (Mizuuchi & Mizuuchi (1980) Proc. Natl. Acad. Sci. U.S.A. 77:3220-4). If the core sequence in attP is replaced with another sequence, and there is an identical sequence on another DNA molecule that can pair with the new core, recombination can also take place. Integration causes the phage chromosome to be covalently attached to the bacterial chromosome, flanked by hybrid sequences called attL and attR. This is the BP reaction, because it involves attB and attP as substrates. The host integration host factor (IHF) is usually also required (Miller et al. (1980) Cell 20:721-9). The BP reaction of bacteriophage lambda requires the attP DNA to be supercoiled but attB DNA can be linear, like a PCR product.

Reversal of the reaction, used to excise lambda from the chromosome, additionally involves the phage protein Xis and restores the original attB and attP sequences. This LR reaction does not require supercoiled DNA and, in fact, works much better when the DNA is relaxed or linear. Both the BP and LR reactions are well characterized, function at room temperature, involve relatively stable proteins, and are robust.

Another site-specific recombination system is the yeast Flp recombinase, which is used in a recombination coupled to replication of the yeast 2μ plasmid (Sadowski (1995) Prog. Nucl. Acid Res. Mol. Biol. 51:53-91). Unlike integrase, the yeast Flp recombinase functions primarily in one direction, catalyzing recombination between two DNA sequences (FRT) that are antiparallel in the plasmid and thereby inverting the sequences between them. In the most common biotechnological applications of Flp/FRT, the FRT sequences are set up in parallel orientation and therefore the reaction leads to excision of the DNA between them (Hoang et al. (1998) Gene 212:77-86; Theodosiou & Xu (1998) Methods 14:355-65). Accordingly, some embodiments of the methods and the vectors of the invention use the lambda or the Flp site-specific recombination systems. The use of other site-specific recombination systems is also within the spirit and scope of the invention.

In a first aspect, the invention provides methods for moving an insert nucleic acid molecule between vectors, comprising transferring an insert nucleic acid molecule from a first vector to a second vector using site-specific recombination in vivo. The methods comprise combining at least three components in vivo: a first vector comprising an insert nucleic acid molecule flanked by at least two recombination sites, a second vector comprising at least two recombination partner sites, and one or more recombination proteins. Some of these components may initially be present in two or more cells. Thus, in some embodiments, the invention further comprises combining one or more plasmid transfer factors, which provide transfer of vectors by conjugation.

In some embodiments, the methods comprise combining two or more cells, wherein each cell comprises at least one of: (a) a first vector comprising a transfer origin and an insert nucleic acid molecule, wherein the insert nucleic acid molecule is flanked by a first recombination site and by a second recombination site; (b) a second vector comprising a transfer origin and a first recombination site partner and a second recombination site partner; (c) one or more recombination proteins, wherein the one or more recombination proteins mediate recombination between the first recombination site and the first recombination site partner, and between the second recombination site and the second recombination site partner; and (d) one or more plasmid transfer factors, which provide inter-cellular transfer of vectors comprising the transfer origin; and wherein the cells are combined under conditions effective to promote conjugation and recombination.

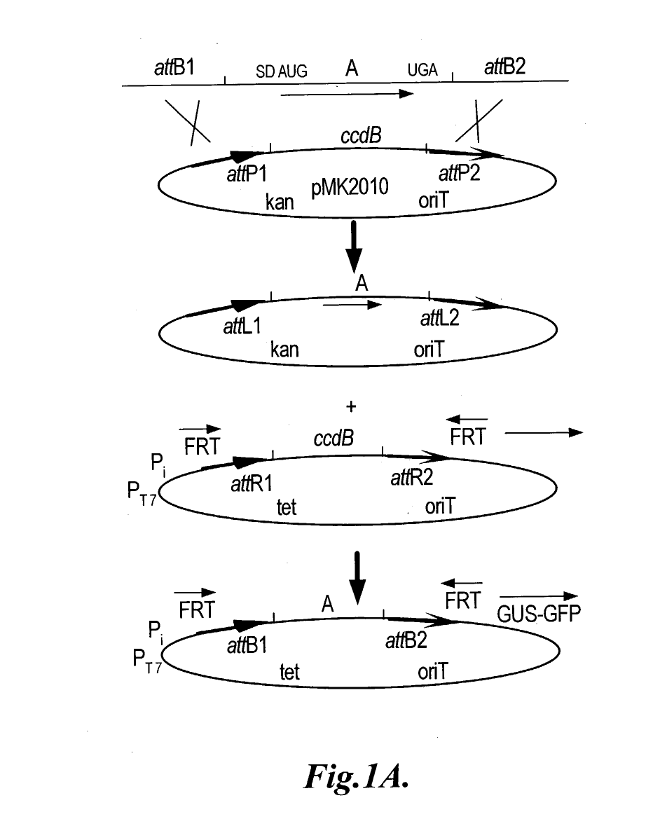

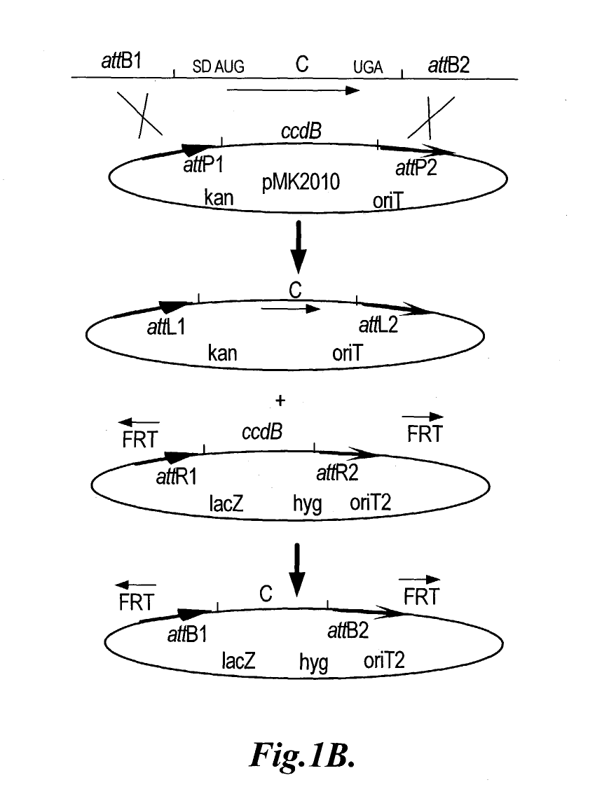

The first and second vectors may be any vector, including vectors which may function in a variety of systems or host cells, for example, prokaryotic vectors, eukaryotic vectors, or vectors which may shuttle between various prokaryotic and/or eukaryotic systems (e.g., shuttle vectors). As used herein, the term “vector” refers to a nucleic acid molecule (preferably DNA) that provides a useful biological or biochemical property to an insert nucleic acid molecule. Examples include plasmids, phages, autonomously replicating sequences, centromeres, and other sequences which are able to replicate or be replicated in vitro or in a host cell, or to convey a desired nucleic acid segment to a desired location within a host cell. A vector may have one or more restriction endonuclease recognition sites at which the sequences can be cut in a determinable fashion without loss of an essential biological function of the vector, and into which a nucleic acid fragment can be inserted in order to bring about its replication and cloning. Vectors may further provide primer sites (e.g., for PCR), transcriptional and/or translational initiation and/or regulation sites, recombinational signals, replicons, selectable markers, etc. Typically, the vectors include one or more selectable markers suitable for use in the identification of cells containing with the vector. Vectors suitable for use in this aspect of the invention are described in FIGS. 1A and 1B, and in EXAMPLE 1.

Prokaryotic vectors for use in the invention include but are not limited to vectors which may propagate and/or replicate in gram negative and/or gram positive bacteria, including bacteria of the genera Escherichia, Salmonella, Proteus, Clostridium, Klebsiella, Bacillus, Streptomyces, Anabaena, Pseudomonas, and Sinorhizobium.

Eukaryotic vectors for use in the invention include vectors which propagate and/or replicate and yeast cells, plant cells, mammalian cells, (particularly human), fungal cells, insect cells, fish cells and the like. Particular vectors of interest include but are not limited to cloning vectors, sequencing vectors, expression vectors, fusion vectors, two-hybrid vectors, and reverse two-hybrid vectors. Such vectors may be used in prokaryotic and/or eukaryotic systems depending on the particular vector.

The vectors used in the methods of the invention typically comprise a transfer origin. The transfer origin allows the transfer of the vector from one cell to another cell by conjugation, in the presence of a helper plasmid. In some embodiments, the transfer origin comprise the oriT sequence from plasmid RK2 or ColE1. Exemplary vectors that may be used in the methods of the invention are the entry vectors and destination vectors described in EXAMPLE and shown in FIGS. 1A and 1B.

The insert nucleic acid molecule may be any nucleic acid molecule derived from any source that may be manipulated by using the methods of the invention, and may include non-naturally-occurring nucleic acid molecules. Typically, the insert nucleic acid molecule is a DNA molecule. The insert nucleic acid molecule may be a population of nucleic acid molecules. An insert nucleic acid molecule may comprise one or more genes or parts of genes.

The first vector comprises the insert nucleic acid molecule flanked by a first and a second recombination site. The second vector comprises a first recombination partner site and a second recombination partner site. The first recombination site recombines with the first recombination partner site, and the second recombination site recombines with the second recombination partner site. The first recombination site and the second recombination site do not substantially recombine with each other. Similarly, the first recombination partner site and the second recombination partner site do not substantially recombine with each other. In some embodiments, the first and second recombination sites (or the first and second recombination partner sites) differ in sequence and do not interact with each other. Alternatively, the first and second recombination sites (or the first and second recombination partner sites) have the same sequence and are manipulated to inhibit recombination with other, for example by introducing a protein binding site adjacent to one of the sites. In the presence of the protein that recognizes the protein binding site, the recombinase fails to access the adjacent recombination site, and the other recombination site is used preferentially.

In some embodiments, the first recombination partner site and the second recombination partner site in the second vector flank a selectable marker, for example, a toxic gene. In some embodiments the toxic gene is ccdB, a gene that is lethal in E. coli strains lacking the antitoxin, ccdA, or a mutated DNA gyrase (Bahassi et al. (1999) J. Biol. Chem. 274:10936-44; Bernard et al. (1993) J. Mol. Biol. 234:534-4). The site-specific recombination event replaces the toxic gene by the insert nucleic acid, and the resulting vector can now be propagated in the appropriate organism without toxic effects.

In the presence of one or more recombination proteins, the insert nucleic acid molecule is moved from the first vector to the second vector by site-specific recombination between the first insert recombination site and the first insert recombination site partner, and the second insert recombination site and the second insert recombination site partner, respectively. As used herein, the term “recombination protein” refers to a protein involved in recombination reactions involving one or more recombination sites. A recombination protein may be an excisive or integrative protein, an enzyme, co-factor or other associated protein. Thus, recombination proteins include, for example, lambda integrase, Integration Host Factor, Xis, and Flp recombinase.

In some embodiments, the first recombination site is an attB1 site and the second recombination site is an attB2 recombination site. In some embodiments, the first recombination partner site is an attP1 site and the second recombination partner site is an attP2 site, and the site-specific recombination is a BP reaction, which catalyzes site-specific recombination between an attB site and an attP site to yield attL and attR sites.

In some embodiments, the product of the recombination between the recombination site and the recombination site partner is a hybrid recombination site that can recombine with a hybrid recombination site partner to yield the original recombination site. In some embodiments, the insert nucleic acid molecule is transferred from a first vector to a second vector by site-specific recombination between a hybrid recombination site in the first vector and a hybrid recombination site partner in the second vector. In some embodiments, the site-specific recombination regenerates the original recombination site flanking the insert nucleic acid molecule in the second vector. In some embodiments, the hybrid recombination site is an attL site, the hybrid recombination partner site is an attR site, and the site-specific recombination is the LR reaction which regenerates an attB recombination site.

In some embodiments, the recombination sites may be modified by one or more mutations to enhance the specificity or the efficiency of the recombination reaction, or to decrease the reverse reaction. Methods for introducing specific mutations into nucleic acid sequences have been previously described (see, e.g., Ausubel et al. (1989-1996) Current Protocols in Molecular Biology, Wiley Interscience, New York).

The one or more recombination proteins may be encoded on a plasmid or they may be encoded by the genome of at least one of the two or more cells. If they are encoded on a plasmid, the plasmid may additionally comprise a transfer origin. In some embodiments, the recombination proteins comprise lambda integrase (Int). Additional, the recombination proteins may comprise Integration Host Factor (IHF) or Xis. Plasmids and genomes encoding Int, IHF, and Xis are available (Platt et al. (2000) Plasmid 43:12-23; Linn & St. Pierre (1990) J. Bacteriol. 172:1077-84).

Plasmid transfer factors permit the transfer of vectors comprising transfer origins from one bacterial cell to another bacterial cell by conjugation. Exemplary plasmid transfer factors are the tra genes of many self-transmissible plasmids (like the F factor or plasmid RK2), or the tra genes acting together with mob functions of mobilizable plasmids (like ColEI). There is generally a specific recognition between the transfer origin DNA sequence, which acts in cis, and the transfer factors, which can act in trans (Heinemann & Sprague (1989) Nature 340:205-9). As described above, both the first vector and the second vector comprise a transfer origin. Thus, the first vector comprising the insert nucleic acid molecule, the second vector, and the one or more recombination proteins may be brought together within one cell for transfer of the insert nucleic acid molecule from the first vector into the second vector by site-specific recombination in vivo. For example, some embodiments of the methods comprise combining a first cell comprising the first vector comprising the nucleic acid insert, and a second cell comprises the second vector. The first or the second cell may additionally comprise one or more recombination proteins and one or more plasmid transfer factors. Some embodiments further comprise combining a third cell comprising one or more recombination proteins. Some embodiments further comprise combining a fourth cell comprising one or more plasmid transfer factors.

Representative cells that are suitable for use in this aspect of the invention are any cells that can exchange genetic material by conjugation. For example, conjugation using the RK2 transfer system has been demonstrated over a wide taxonomic range, including gram negative bacteria, cyanobacteria, lower eukaryotes and mammalian cells (see, e.g., Waters (2001) Nat. Genet. 29:375-6).

Conditions effective to promote conjugation and recombination comprise any conditions that are suitable for transferring vectors between cells by conjugation and for permitting in vivo recombination to take place. Generally, conditions effective to promote conjugation and recombination comprise any conditions compatible with the growth of the cells comprising the vectors, the recombination protein(s), and/or the plasmid transfer factor(s), and are typically the normal growth conditions of these cells. Thus, if the cells are E. coli cells, suitable conditions comprise standard growth media and temperatures, such as LB broth and a temperature of around 37° C., as described in EXAMPLES 2 and 3. Other temperature conditions, such as for example 30° C. or room temperature may also be suitable for conjugation and recombination in E. coli. Conditions effective to promote conjugation and recombination depends on the cells used. For example, 4° C. may be a suitable temperature for conjugation and recombination using cold-adapted Vibrio.

Some embodiments further comprise combining a fifth cell that allows for selection of the second vector comprising the insert nucleic acid molecule, as described in EXAMPLES 2 and 3. Typically, the first and second vector comprise at least one selectable marker. A selectable marker may be used to select for or against a cell that contains it. Examples of selectable markers include, for example, DNA segments that encode products which provide resistance against otherwise toxic compounds (e.g., antibiotics), DNA segments that encode products which are otherwise lacking in the recipient cell (e.g., tRNA genes, auxotrophic markers), DNA segments that encode products which suppress the activity of a gene product, DNA segments that encode products which can be readily identified (e.g., phenotypic markers such as β-galactosidase, green fluorescent protein (GFP), and cell surface proteins), DNA segments that bind products which are otherwise detrimental to cell survival and/or function, DNA segments that otherwise inhibit the activity of a gene (e.g., antisense oligonucleotides), DNA segments, which when absent, directly or indirectly confer resistance or sensitivity to particular compounds, and/or DNA segments that encode products which are toxic in recipient cells (e.g., ccdB).

In some embodiments, the methods of this aspect of the invention comprise combining: (a) a first cell comprising a first vector comprising a transfer origin and an insert nucleic acid molecule, wherein the insert nucleic acid molecule is flanked by a first recombination site and by a second recombination site; (b) a second cell comprising a second vector comprising a transfer origin and a first recombination site partner and a second recombination site partner; (c) a third cell comprising one or more recombination proteins, wherein the one or more recombination proteins mediate recombination between the first recombination site and the first recombination site partner and between the second recombination site and the second recombination site partner; and (d) a fourth cell comprising one or more plasmid transfer factors, which provide inter-cellular transfer of vectors comprising the transfer origin; wherein the cells are combined under conditions effective to promote under conditions effective to promote conjugation and recombination. Some embodiments further comprise combining a fifth cell that allows for selection of the second vector comprising the insert nucleic acid molecule. Exemplary methods of this aspect of the invention combining 5 different cell types (penta-parental combinations) are described in EXAMPLES 2 and 3.

Using the methods of the invention, one or more insert nucleic acid molecules, such as a population of insert nucleic acid molecules, may be transferred into any number of vectors. Thus, for example, a mixed population of inserts (prepared by mutagenesis, for example) could be transferred into a second vector en masse prior to screening for some selectable property in the new vector context. An insert nucleic acid molecule may be transferred into one or more vectors in one experimental manipulation. Thus, for example, the second vector may comprise a plurality of vectors, and one or more insert nucleic acid molecules may be transferred into a plurality of second vectors in the same mating mixture by carrying out several conjugations simultaneously. An insert nucleic acid molecule may also be transferred to any number of vectors sequentially, for example, by first transferring the insert nucleic acid molecule from the first vector into the second vector, and then transferring the insert nucleic acid molecule from the second vector into a third vector. Thus, transfers of insert nucleic acid molecules may be accomplished separately, sequentially, in parallel, or en masse. Accordingly, the methods of the invention are particularly suited for high throughput applications and for methods using genetic selection of desired recombinants.

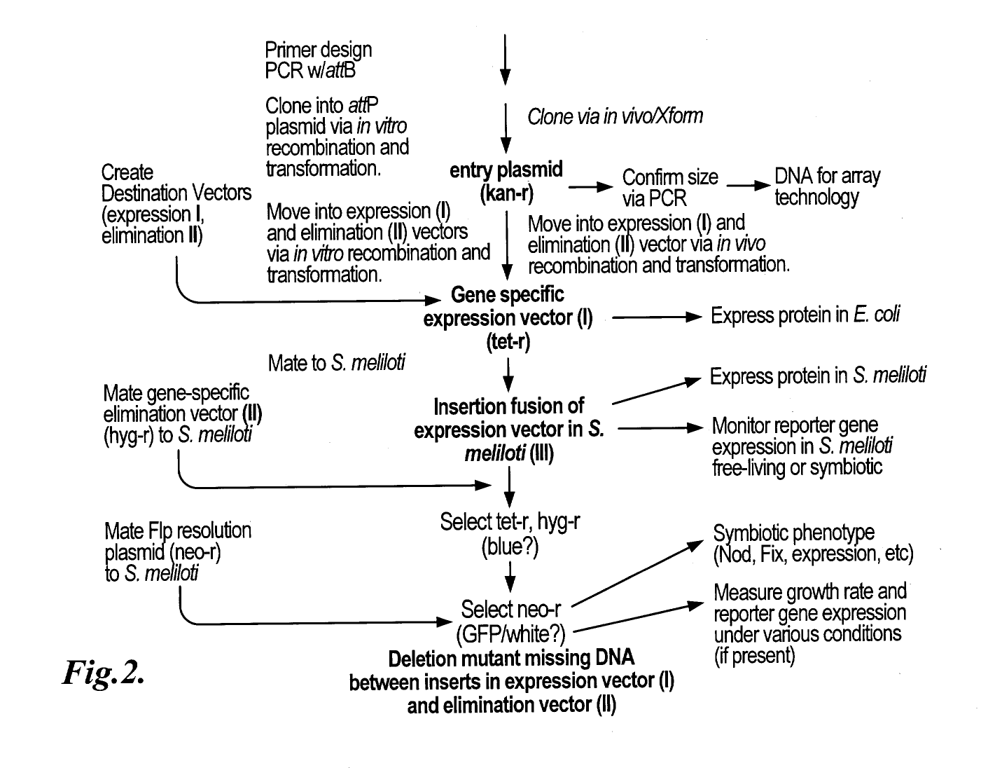

In a second aspect, the invention provides methods and vectors for analyzing a genome using site-specific recombination in vivo, as shown in FIG. 2 In some embodiments, the methods comprise (a) providing a first vector comprising a transfer origin and an insert nucleic acid coding molecule flanked by a first recombination site and by a second recombination site, wherein the insert nucleic acid molecule comprises a sequence from a genomic region in a first organism; (b) transferring the insert nucleic acid molecule within the first vector into a second vector comprising a transfer origin and a first recombination site partner and a second recombination site partner by site-specific recombination in a second organism; (c) transferring the second vector from the second organism into the first organism by inter-organismal transfer; and (d) analyzing the function of the genomic region in the first prokaryotic organism. Typically, the first and second organisms are different prokaryotic organisms.

In some embodiments of this aspect of the invention, the first vector is an entry vector. In some embodiments, the entry vector is created by introducing the insert nucleic acid molecule into a donor vector using site-specific recombination, as described in EXAMPLE 1. In some embodiments, the second vector is a destination vector, as described in EXAMPLE 1. Accordingly, in step (b) of the methods, the insert nucleic acid molecule is transferred from an entry vector to a destination vector by site-specific recombination in vivo, as described above for the first aspect of the invention and in EXAMPLES 2 and 3.

In some embodiments, the first vector and the second vector are destination vectors, and the insert nucleic acid molecule is transferred from one destination vector to another destination vector by site-specific recombination in vivo, as described above for the first aspect of the invention and in EXAMPLES 2 and 3. The donor, entry, and destination vectors of the invention include vectors which may function in a variety of systems or host cells, for example, prokaryotic vectors, eukaryotic vectors, or vectors which may shuttle between various prokaryotic and/or eukaryotic systems (e.g., shuttle vectors).

Prokaryotic vectors for use in the invention include but are not limited to vectors which may propagate and/or replicate in gram negative and/or gram positive bacteria, including bacteria of the genus Escherichia, Salmonella, Proteus, Clostridium, Klebsiella, Bacillus, Streptomyces, Pseudomonas, and Sinorhizobium.

The insert nucleic acid molecules used in accordance with the invention are flanked by two or more recombination sites which allow the insert nucleic acid molecule to be transferred or moved into one or more vector molecules in accordance with the invention. The insert nucleic acid molecules of the invention may be prepared by any number of techniques by which two or more recombination sites are added to the molecule of interest. Such means for including recombination sites to prepare the insert nucleic acid molecules of the invention includes mutation of a nucleic acid molecule (e.g., random or site specific mutagenesis), recombinant techniques (e.g., ligation of adapters or nucleic acid molecules comprising recombination sites to linear molecules), amplification (e.g., using primers which comprise recombination sites or portions thereof), transposition (e.g., using transposons which comprise recombination sites), recombination (e.g., using one or more homologous sequences comprising recombination sites), nucleic acid synthesis (e.g., chemical synthesis of molecules comprising recombination sites or enzymatic synthesis using various polymerases or reverse transcriptases.) and the like. In accordance with the invention, nucleic acid molecules to which one or more recombination sites are added may be any nucleic acid molecule derived from any source and may include non-naturally occurring nucleic acids. Additionally, the nucleic acid molecules of interest for producing insert nucleic acid molecules may be linear or circular and further may comprise a particular sequence of interest (e.g., a gene) or may be a population of molecules (e.g., molecules generated from a genomic or cDNA libraries).

In some embodiments, libraries (e.g., populations of genomic DNA or cDNA, or populations of nucleic acid molecules produced by de novo synthesis, such as random sequences or degenerate oligonucleotides) are utilized in accordance with the present invention. By inserting or adding recombination sites to such populations of nucleic acid molecules, a population of insert nucleic acid molecules is produced. By the recombination methods of the invention, the library may be easily moved into different vectors (or combinations of vectors) and thus into different host systems (prokaryotic and eukaryotic) to evaluate and analyze the library or a particular sequences or clones derived from the library. Alternatively, the vectors containing the desired molecule may be used in in vitro systems, such as in vitro expression systems for production of RNA and/or protein.

In some embodiments of the invention, the insert nucleic acid molecules are flanked by a first insert recombination site and a second insert recombination site. In some embodiments, the first insert recombination site is an attB1 site and the second insert recombination site is an attB2 site (see EXAMPLE 1). These insert nucleic molecules are then introduced into a donor vector containing a toxic gene flanked by a first and a second insert recombination site partner to yield an entry vector by site-specific recombination between the first insert recombination site and the first insert recombination site partner, and the second insert recombination site and the second insert recombination site partner, respectively. In some embodiments, the product of the recombination between the recombination site and the recombination site partner is a hybrid recombination site that can recombine with a hybrid recombination site partner to yield the original recombination site. In some embodiments, the first insert recombination site partner is an attP1 site, and the second insert recombination site partner is an attP2 site, and the site-specific recombination is a BP reaction, which catalyzes site-specific recombination between an attB site and an attP site to yield an attL site.

In some embodiments the toxic gene is ccdB, a gene that is lethal in E. coli strains lacking the antitoxin, ccdA, or a mutated DNA gyrase (Bahassi et al. (1999) J. Biol. Chem. 274:10936-44; Bernard et al. (1993) J. Mol. Biol. 234:534-4). The site-specific recombination event replaces the toxic gene by the insert nucleic acid, and the resulting vector can now be propagated in the appropriate organism without toxic effects.

In some embodiments, the insert nucleic acid sequences are transferred from an entry vector to a destination vector by site-specific recombination between a hybrid recombination site in the entry vector and a hybrid recombination site partner in the destination vector. In some embodiments, the site-specific recombination regenerates the original recombination site flanking the insert nucleic acid molecule in the destination vector. In some embodiments, the hybrid recombination site is an attL site, the hybrid recombination partner site is an attR site, and the site-specific recombination is the LR reaction which regenerates an attB recombination site.

Embodiments of destination vectors include expression vectors, elimination vectors, and replicating vectors (see EXAMPLE 1). Other embodiments of the invention include the use of insert nucleic acid sequences with hybrid recombination sites and entry vectors with hybrid recombination site partners, wherein the recombination between the hybrid recombination site and the hybrid recombination site partners generates a recombination site, which can recombine with a recombination site partner in a destination vector to generate a hybrid recombination site.

In some embodiments, the insert nucleic acid molecules are transferred into a vector containing recombination sites for an additional recombination system, such as the FRT/Flp system. In some embodiments, the vectors contain at least one selectable marker. In some embodiments, the vectors contain at least one reporter gene, such as β-glucoronidase (GUS) or green fluorescent protein (GFP), or fusions between GUS and GFP.

The first prokaryotic organism may be any desired prokaryotic organism, including Escherichia, Salmonella, Proteus, Clostridium, Klebsiella, Enterococcus, Bacillus, Streptomyces, Anabaena, Pseudomonas, Sinorhizobium, and other bacterium able to serve as recipients in conjugation. The second prokaryotic organism may be any organism capable of supporting recombinase-mediated recombination. The second prokaryotic organism is typically E. coli, although other bacteria could be used in situations in which the conditions needed for the second organism to conjugate with the first organism are incompatible with growth of E. coli (for example, low or high temperatures).

In the third step of this aspect of the invention, the second vector comprising the insert nucleic acid molecule is transferred from the second prokaryotic organism back into the first prokaryotic organism. Typically, the second vector comprising the insert nucleic acid molecule is transferred by conjugation. For example, if the first prokaryotic organism is S. meliloti and the second prokaryotic organism is E. coli, transfer of the vector into S. meliloti uses the established conjugation technique of triparental mating (Ditta et al. (1980) Proc. Natl. Acad. Sci. U.S.A. 77:7347-5).

In the fourth step of this aspect of the invention, the function of the genomic region in the first prokaryotic organism is analyzed. These analyses comprise expressing the insert nucleic acid molecule in the first prokaryotic organism (sense or antisense), monitoring expression of the insert nucleic acid by operably linking it to a reporter gene, creating deletions of the genomic region (see below), complementing mutations in the first prokaryotic organism, etc. Representative methods of analyzing the function of the genomic region in the first prokaryotic organism are provided in EXAMPLE 1. In some embodiments, the analysis of the genomic region comprises introducing the insert nucleic acid molecule within the second vector into the genome of the first prokaryotic organism by homologous recombination, as described in EXAMPLE 1.

In a third aspect, the invention provides methods for deleting a target region in a prokaryotic genome by site-specific recombination in vivo. The methods comprise: (a) introducing a first recombination site into a first genomic region by homologous recombination, wherein the first genomic region is adjacent to a first end of a target genomic region; (b) introducing a second recombination site into a second genomic region by homologous recombination, wherein the second genomic region is adjacent to a second end of the target genomic region; and (c) deleting the target genomic region by providing one or more recombination proteins to catalyze site-specific recombination between the first and second recombination sites. In some embodiments, the excised target genomic region is recovered as a reciprocal recombination product.

In some embodiments, step (a) comprises introducing a first vector comprising a first DNA sequence and a first recombination site into a first genomic region by homologous recombination, wherein the first genomic region is adjacent to a first end of a target genomic region and the first DNA sequence is homologous to the first genomic region; and step (b) comprises introducing a second vector comprising a second DNA sequence and a second recombination site into a second genomic region by homologous recombination, wherein the second genomic region is adjacent to a second end of a target genomic region, the second DNA sequence is homologous to the second genomic region, and the second recombination site has the same orientation as the first recombination site. Thus, a first insert nucleic acid molecule homologous to a first genomic region is introduced into a first destination vector (e.g., an expression vector, as described in EXAMPLE 1 and FIG. 1A) adjacent to a first recombination site. The first recombination site is introduced into the genome by homologous recombination between the first insert nucleic acid molecule in the first destination vector and the first genomic region. A second insert nucleic acid molecule is introduced into a second destination vector (e.g., an elimination vector, as described in EXAMPLE 1 and FIG. 1B) adjacent to a second recombination site. The second recombination site is introduced into the genome by homologous recombination between the second insert nucleic acid molecule in the second destination vector and the second genomic region. A recombinase is then provided to catalyze recombination between the first and second recombination sites resulting in the deletion of the target genomic region between the first and the second genomic region.

In some embodiments, step (a) comprises homologously recombining a first vector comprising a first DNA sequence between a first and a second recombination site into a first genomic region, wherein the first genomic region is adjacent to a first end of a target genomic region, the first DNA sequence is homologous to the first genomic region, and the first and second recombination sites have different orientations; and step (b) comprises homologously recombining a second vector comprising a second DNA sequence between a third and fourth recombination site into a second genomic region, wherein the second genomic region is adjacent to a second end of the target genomic region, the second DNA sequence is homologous to the second genomic region, the third recombination site has the same orientation as the second orientation site, and the fourth recombination site has the same orientation as the first site-specific recombination site. Thus, the first insert nucleic acid molecule is flanked by a first and second recombination site in inverted orientation (e.g., directed away from the insert nucleic acid molecule, as shown in FIG. 1B), and the second insert nucleic acid molecule is flanked by a third and fourth site-specific recombination site in inverted orientation (e.g., directed towards the insert nucleic acid molecule, as shown in FIG. 1A), such that the first and fourth site-specific recombination site are in parallel orientations and the second and the third site-specific recombination sites are in parallel orientations opposite to the orientation of the first and fourth site-specific recombination sites. The action of a site-specific recombination system on the second and third parallel sequences will lead to the deletion of the target genomic region between the first and the second genomic region from the chromosome, and the generation of an extrachromosomal plasmid containing the target genomic region and vector sequences. The extrachromosomal plasmid containing the target genomic region and vector sequences may be isolated. In some embodiments, the recombination system used for deleting a target genomic region from the chromosome and generating the vector containing the target genomic region is the FRT/Flp system.

In some embodiments, the identities of the first and second genomic regions flanking the target genomic region are previously known. Alternatively, nucleic acid sequences corresponding to the first and second genomic regions are first isolated by standard molecular cloning techniques using the target genomic region to probe for adjacent nucleic acid sequences.

In a fourth aspect, the present invention provides an integrated and extendable set of site-specific recombination vectors that can be used for functional analyses of genomes. In some embodiments, the invention provides donor vectors containing site-specific recombination sites. In some embodiments, the invention provides destination vectors containing site-specific recombination sites.

In some embodiments, the vectors of the invention comprise a first and a second recombination site flanking a selectable marker (e.g., a toxic gene), one or more selectable markers, and a transfer origin for conjugation. In some embodiments, the selectable marker is a toxic gene. The vector may further comprise additional selectable markers. In some embodiments, the first and second recombination sites are att sites. In some embodiments, the transfer origin comprises the oriT sequence from plasmid RK2 or ColEI. In some embodiments, the vector comprises pMK2010 (SEQ ID NO:1).

In some embodiments, the vectors of the invention use two related site-specific recombination systems that are involved in lambda integration and in yeast plasmid replication. These recombination systems have been adapted to carry out highly directed rearrangements in vitro and in vivo. In some embodiments, the vectors of the invention allow the cloning of predicted ORFs of a genome and other interesting genes, such as tRNAs, using high-throughput, genomic scale PCR techniques. The vectors allow the clones to be used immediately in creating DNA arrays for expression analysis and facilitate the creation of reporter genes fused to each predicted promoter as well as deletions of target genomic regions.

In some embodiments, the vectors of the invention can be used for the functional analysis of a bacterial genome. In some embodiments, the bacterial genome is the genome of Sinorhizobium meliloti. In some embodiments, the. vectors of the invention are used (1) to clone the open reading frames (ORFs) and other genes into a donor vector using integrase mediated recombination to generate entry vectors, (2) to amplify all open reading frames from the entry vectors and construct DNA microarrays for direct analysis of mRNA expression for profiling genomic DNA from other rhizobia to determine how representative the sequence is, (3) to recombine these entry vectors with other vehicles and construct sets of destination vectors that can be used for monitoring gene expression through reporter technologies, for generating nucleic acid probes and for producing proteins, and (4) to generate deletions in the S. meliloti genome covering individual ORFs and regions of 10-20 ORFs. Accordingly, instead of separating various technical approaches to the biology of S. meliloti such as array construction, reporter gene fusions, mutagenesis and protein production and characterization, the vectors of the invention provide a platform in which it is possible to move smoothly between different levels of genetic and biochemical analysis (see FIG. 2). An advantage of this experimental design is an imbedded flexibility that enables the next new idea to build with these materials rapidly and inexpensively. Thus, the vectors of the invention provide a practical and efficient system to carry out a set of standard constructions on a genome scale that will yield valuable information and will serve as a platform for further investigations.

In a fifth aspect, the invention provides site-specific recombination kits for the systematic functional analysis of a genome. In some embodiments, the kits comprise one or more vectors of the invention in combination with instructions for using the vector(s) in one or more methods of the invention. For example, the kits may provide instructions for moving one or more insert nucleic acid molecules from a first vector into a second vector using site-specific recombination in vivo. In some embodiments, the kits further comprise cells suitable for in vivo recombination, and instructions for using the cells in one or more methods of the invention. For example, the kits may comprise cells comprising coding sequences for recombination proteins and/or plasmid transfer factors. The kits may also comprise cells that allow for selection of recombinant second vectors comprising the insert nucleic acid molecule(s). In some embodiments, the kits further comprise one or more specialized apparati for carrying out the manipulations, for example, the replicator described in EXAMPLE 3, and instructions for using the replicator in one or more methods of the invention.

### EXAMPLE 1

This example describes representative methods and vectors for the systematic functional analysis of a genome, for example, the S. meliloti genome.

1. Rhizobium Genome Structure and Genetics

Rhizobia have various genome arrangements (Hynes & Finan (1998) in The Rhizobiaceae (Spaink et al., eds.) pp. 25-43). B. japonicum has a single large chromosome, M loti has a large chromosome -and two relatively small plasmids, and many rhizobia have multiple plasmids (Hynes & McGregor (1990) Mol. Microbiol. 4:567-574). S. meliloti 1021 has a 3.65 Mb chromosome and two large megaplasmids, pSymA (1.35 Mb) and pSymB (1.68 Mb). The nucleotide sequence of the S. meliloti 1021 genome has been determined (Galibert et al. (2001) Science 293(5530):668-72).

The three replicons in S. meliloti 1021 were predicted to contain 6204 open reading frames (ORFs) with an average length of 309 amino acids. Most essential genes are located on the chromosome but an arginine tRNA on pSymB is needed to complete the set needed for translation. pSymB also has a minCDE operon likely to be essential for cell division control. pSymB regions that contain these genes can not be deleted (Chain et al. (2000) J. Bacteriol. 182. 5486-5495). pSymA carries many functions known to be involved in symbiosis but, consistent with the ability to cure pSymA (Oresnik et al. (2000) J. Bacteriol. 182:3582-3586), no essential functions are unique to pSymA. 3704 of the 6204 ORFs predicted originally resemble known proteins enough to make a functional assignment. About 500 ORFs had no match in the databases, and the rest (the “conserved hypothetical” proteins) resemble ORFs from other organisms that had no established function. Further analysis is required to determine the validity of these functional assignments and to determine if the hypothetical ORFs represent real proteins.

S. meliloti has the most advanced bacterial genetics of any Rhizobium species (Hynes & Finan (1998) in The Rhizobiaceae (Spaink et al., eds.) pp. 25-43). There are suicide plasmids with several different antibiotic resistance markers, various plasmid replicons that can be used to maintain genes, and transposons useful for generating various kinds of transcriptional and translational fusions and for inserting regulated promoters into the S. meliloti genome (Barnett et al. (2000) Biotechniques 29:240-5; Blatny et al. (1997) Plasmid 38:35-51). Most current approaches use conjugation as a method of getting DNA into S. meliloti since, while electroporation can be used to introduce DNA into S. meliloti 1021, the efficiency is relatively low. A generalized transducing phage is widely used (Finan et al. (1984) J. Bacteriol. 159:120-4).

2. Methods of Analyzing the S. meliloti Genome

High-throughput, genomic-scale PCR techniques are used to clone the predicted S. meliloti ORFs and other interesting genes, such as tRNAs. These clones may be used immediately for creating DNA arrays for expression analysis. They also allow the creation of S. meliloti strains with reporter genes fused to each predicted promoter and the isolation of deletion strains missing precisely defined regions of the S. meliloti genome. As shown in FIG. 2, these clones can be used to investigate important questions related to symbiosis.

a. Entry Vectors and PCR Strategy. PCR products are cloned into a donor vector to create an entry vector. The donor vector has two attP sites, attP1 and attP2, flanking ccdB. ccdB is a toxin gene that is lethal in E. coli strains lacking the antitoxin, ccdA, or a mutated DNA gyrase (Bahassi et al. (1999) J. Biol. Chem. 274:10936-44; Bernard et al. (1993) J. Mol. Biol. 234:534-4). An exemplary donor vector, pMK2010, is shown in FIGS. 1A and 1B. pMK2010 was created by inserting an oriT sequence from plasmid RK2 as an SphI fragment from plasmid pBSL237 (Alexeyev & Shokolenko (1995) Biotechniques 19: 22-4) into the NspI site of plasmid into pDONR201 (GATEWAY, Invitrogen Life Technologies, Inc.). oriT is a transfer origin and its presence allows a replicon containing it to participate in conjugation in the presence of appropriate tra-and mob-encoded accessory proteins. The DNA sequence of pMK2010 is provided in SEQ ID NO:1.

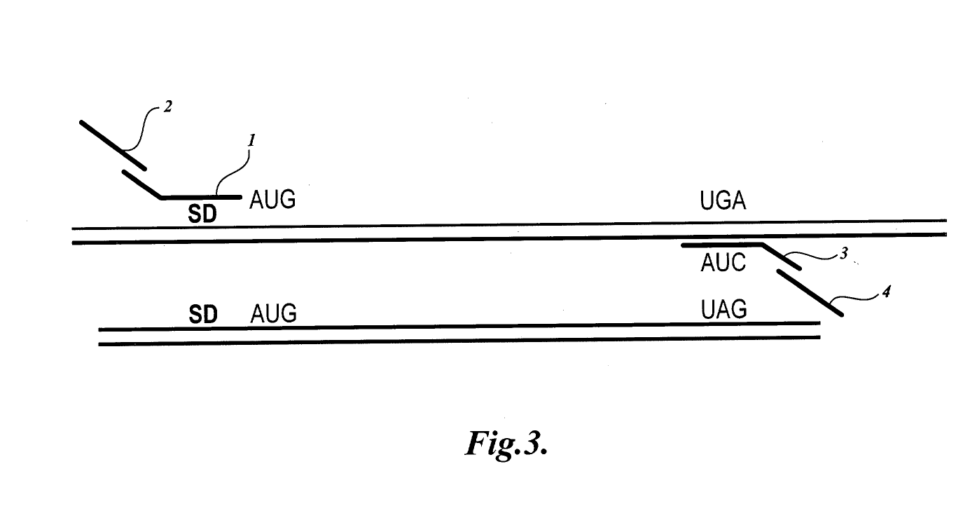

DNA inserts are synthesized using PCR primers that contain the attB1 and attB2 sites. Nested PCR can be performed, starting with first primers containing only 12 bp of the attB sequence in the first rounds of synthesis, then amplifying the products from the first round of synthesis using second primers that contain the entire attB sequence (FIG. 3). The second set of primers is standard and thus less expensive.

Open reading frames to be cloned are based on bioinformatic predictions. First forward primers at the start of the open reading frame are selected to include the first 20 bases of the gene predicted to code for the protein, with the initiation codon changed to ATG if this codon is not already used. 5′ to the ATG, the first forward primers include the sequence 5′-GGAGGCTCTTCA-3′ (SEQ ID NO:2), which contains a region of similarity to the attB forward second primer, and a ribosome binding site predicted to be relatively strong in S. meliloti operatively linked to the ATG. Additional features in the first forward primers are restriction sites to facilitate further manipulation and a length suitable for placing the ORF to be cloned in frame with N-terminal fusion destination vectors (described below). The second forward primer is 5′-GGGGACAAGTTTGTACAAAAAAGCAGGCTTAGGAGGCTCTTCAATG-3′ (SEQ ID NO:3).

First reverse primers at the carboxyl terminus of the ORF also have 20 bases of homology with the end of the ORF, 3′ to the sequence 5′-AGCTGGGTTCTA-3′ (SEQ ID NO:4), where the CTA in this sequence is present to substitute a UAG stop codon for the one normally found at the end of the coding region and to be phased so that translation will be able to read through into C-terminal fusion tags when a strong suppressor is introduced (O'Connell et al. (2000) Appl. Environ. Microbiol. 66:392-400). The second reverse primer used is 5′-GGGGACCACTTTGTACAAGAAAGCTGGGTTCTA-3′ (SEQ ID NO:5).

The PCR reaction is carried out for several rounds (typically 20 rounds) with gene-specific first primers, then the attB-containing second primers are added and the PCR reaction is run to completion, typically for an additional 25-30 cycles. PCR is performed with a KOD proofreading polymerase (EMD Biosciences) to keep the error rate low. Other proofreading polymerases, such as the Pfx polymerase (Cline et al. (1996) Nucl. Acids Res. 24:3546-51) may also be used. To decrease the problem of incorrect priming, a “hot start” polymerase that is blocked by an antibody (Kellogg et al. (1994) Biotechniques 16:1134-7) is used. Thus, PCR reactions are set up at room temperature and when the temperature is raised, the antibody is denatured and the reaction begins. DNA in PCR reactions is monitored using gel electrophoresis and fluorescence. DMSO and glycerol are used in the PCR reactions to reduce problems caused by high G+C DNA (Dutton et al. (1993) Nucl. Acids Res. 21:2953-4; Moreau et al. (1997) Methods Mol. Biol. 67:47-53; Varadaraj & Skinner (1994) Gene 140:1-5).

If necessary, PCR products are purified by polyethylene glycol precipitation. Aliquots of the PCR products are mixed with pMK2010 and BP reaction components, incubated at room temperature, then treated with proteinase K to remove integrase. DNA is transformed or electroporated into competent E. coli at high frequency. Recombination replaces ccdB (FIGS. 1A and 1B, top) and the resulting DNA can transform ccdA-minus E. coli. Several clones from each construction can be saved as insurance against the imprecision of PCR. Alternatively, the remaining DNA from the BP reaction may be saved.

The BP reaction is typically performed in vitro, as previously described (see Instruction Manual, GATEWAY, Invitrogen Life Technologies, Inc.). Immediately after the BP reaction, it is possible to make further constructions and transfer the PCR DNA into destination vectors in vitro. However, in vivo recombination is typically used to transfer the PCR products from entry vectors into destination vectors, as described in EXAMPLES 2 and 3.

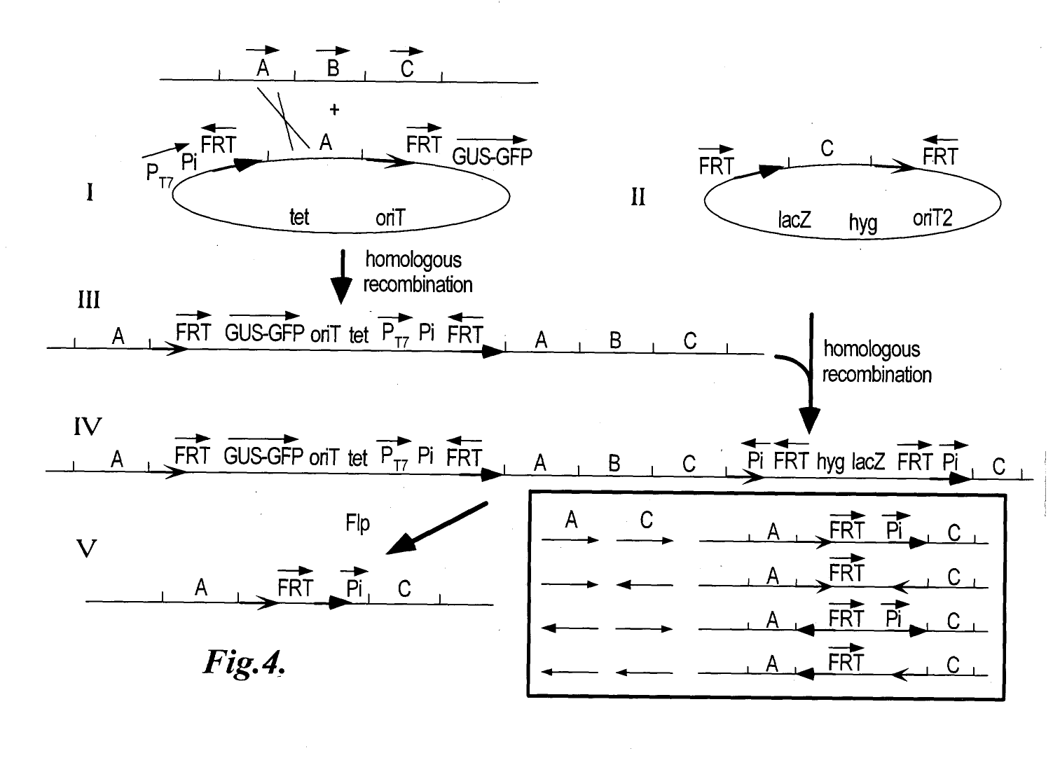

b. Destination Vectors. The insertion of the PCR product into the donor vector to produce an entry vector places it between two attL sites. Integrase-catalyzed recombination is used to exchange this inserted DNA into a destination vector, such as an expression vector, an elimination vector, or a replicating vector. The first two of these vectors are unable to replicate in S. meliloti and are designed to be integrated into the genome, where they can be used to monitor gene expression, produce limited quantities of specific protein and serve as endpoints for constructed deletions. The third has a broad host range replicon and is used for complementation studies and expression of proteins. FIGS. 1A and 1B show the transfer of the cloned DNA into two destination vectors: into an expression vector (FIG. 1A) and into an elimination vector (FIG. 1B). FIG. 4 shows the use of these two nonreplicating vectors to create transcriptional fusions and deletions.

Expression Vectors: The expression vector (FIGS. 1A, FIG. 4, left) contains reporter genes downstream of the cloned ORF oriented so that, after. recombination into the host genome, expression of these genes is under control of the sequences that control expression of the ORF (FIG. 4, III). Two reporter genes that have been used in S. meliloti, β-glucuronidase (GUS) and green fluorescent protein (GFP), have been fused to each other in both the GUS-GFP and GFP-GUS orientations (Quaedvlieg et al. (1998) Plant Mol. Biol. 38:861-73). Both have some advantages and some disadvantages. β-glucuronidase and similar enzymes like β-galactosidase are more sensitive because they are catalytic. They have been used to follow gene expression in free-living cells and bacteroids and have been used very effectively to monitor the pattern of gene expression in nodules (see, e.g., Soupene et al. (1995) Proc. Natl. Acad. Sci. U.S.A. 92:3759-63). GFP and its variants (Scholz et al. (2000) Eur. J. Biochem. 267:1565-70; Yang et al. (1998) J. Biol. Chem. 3;273:8212-6) have the advantage that they can be detected directly but their fluorescence may not be bright enough for some purposes since nodule cells have low background fluorescence.

Other feature optionally present in the expression vector are transcription terminators to protect the plasmid replication and an RNase III cleavage site to produce a signal that is more indicative of promoter strength (Linn & St. Pierre (1990) J. Bacteriol. 172:1077-84). The expression vector also has an antibiotic resistance determinant that can be selected in S. meliloti, and a replication origin that is functional in E. coli, but not in S. meliloti, so that incorporating the antibiotic resistance into S. meliloti requires homologous integration into the S. meliloti genome. There are many distinct replicons of this type (Barnett et al. (2000) Biotechniques 29:240-5; Blatny et al. (1997) Plasmid 38:35-51; Quandt & Hynes (1993) Gene 127:15-21). Transfer of the vector into S. meliloti uses the established conjugation technique of triparental mating (Ditta et al. (1980) Proc. Natl. Acad Sci. U.S.A. 77:7347-5) so an origin of transfer is required. In order for recombinants carrying the ORF to be selected, the vector must contain attR sequences flanking a gene that is toxic. Several toxic genes have been described (Gabant et al. (2000) Biotechniques 28:784-8), including sacB and the ccdB gene (Bernard et al. (1993) J. Mol. Biol. 234:534-4).

The expression vector may also include controllable promoters active in E. coli and S. meliloti. These allow expression of the protein from the replicating vector in E. coli and from the integrated vector in S. meliloti. For E. coli, the T7 promoter is the promoter of choice since it allows high levels of protein expression (Hoang et al. (1999) Gene 237:361-71). In Pseudomonas the T7 promoter is active after the addition of a plasmid that expresses T7 RNA polymerase (Hoang et al. (2000) Plasmid 43:59-72). Alternatively, an S. meliloti α-galactosidase (melibiose)-inducible promoter for driving gene expression has been developed (Bringhurst & Gage (2000) FEMS Microbiol. Lett. 188:23-27).

The expression vector also contains two yeast FRT DNA sequences in inverted orientation. These will allow Flp-catalyzed recombination to create deletion mutations (described below). The locations of the ° FRT sites in the vector determine how much DNA is left behind after the excision occurs and, if it is desired to leave a reporter gene behind after deletion, this may be accomplished by placing the reporter genes outside of the inverted FRT sequences.

After recombination with an entry vector containing a specific gene, A, the A-specific expression vector is conjugated into S. meliloti, selecting for antibiotic resistance (FIG. 4, III). This leads to strains that have duplicate copies of A. The regulatory sequences controlling one of these now directs the expression of reporter genes; a controllable promoter will now regulate the expression of the other copy of A. At this stage there is no “mutation” in the sense that all coding sequences are intact and a copy of A is under control of its normal promoter. If polarity on other genes (B, C) creates a problem, the regulation of expression of these using the controllable promoter should boost the recovery of recombinants.

Elimination Vectors: A second type of destination vector has a replicon active in E. coli, but not in S. meliloti, an origin of transfer, an antibiotic resistance marker different from that used in the expression vector, and a pair of yeast FRT sequences arranged in inverted orientation flanking the attR sites but in an orientation opposite that in the expression vector (FIGS. 1B and 4). A controllable promoter is included between the FRT sequences and the att sequences. To delete gene (or region) B, which is flanked by genes A and C, an elimination vector containing gene C is conjugated into a strain that already contains an integrated copy of the expression vector that carries gene A. Selection is for both antibiotic resistances. A plasmid carrying the yeast Flp gene is introduced into this intermediate strain (IV). Expression of the Flp recombinase results in a deletion (V) between the most distant parallel FRT sequences, including the region from the end of A to the beginning of the structural gene of C. The precise sequences that would remain are determined by the original orientation of the open reading frames (box at lower right). The controllable promoter inserted between FRT and the attR sequences in the elimination vector is retained in the final construct when C is read to the right but is not needed when C is read to the left. Cartridges that have all four orientations of FRT sequences flanking an attB1-Cam-ccdB-attB2 cassette have been created. With other orientations of FRT sequences, it is possible to clone the DNA between two similar insertions as the reciprocal recombinant.

The elimination vector has as little sequence homology with the expression vector as possible to prevent the elimination vector from recombining into the expression vector. Homology of the att sequences and FRT sequences is unavoidable in this implementation, but these sequences are much shorter than is generally thought to be needed for homologous recombination, and recombination into the genome is more likely to occur in the larger region of ORF DNA carried by the elimination vector. The pRK2073 plasmid used to mobilize both the elimination vector and the expression vector in the usual triparental mating carries mobilization sequences for both plasmids RK2 and ColE1, so one destination vector carries oriT-RK2 and the other carries the non-homologous oriT-ColE1. The deletion strategy makes it possible to determine if any gene in the interval is essential. If a merodiploid strain containing both the expression and elimination vectors can be constructed, it is possible to delete the intervening DNA unless it contains an essential gene.

Initially, a set of strains that contain deletions of short regions comprising 10-20 genes is generated in order to make it possible to screen 300-600 strains in a first step. For example, to test regions of pSymA for their importance on different host plants, it would be possible to work with perhaps 60-100 regional deletion strains, find those that had a different phenotype on different host plants, such as Medicago sativa or Medicago truncalula, and subsequently determine which genes covered by the deletions are responsible for the phenotype.

Replicating Vectors: The S. meliloti ORFs are also recombined into a vector that resembles the expression vector but which has a replicon that can function in S. meliloti and carries a different reporter gene, like lacZ, which codes for β-galactosidase. One major use of the derivatives of this vector is to complement mutants isolated in S. meliloti, especially mutants that might be obtained by chemical mutagenesis. For example, if a mutant was isolated that was unable to grow in low calcium concentrations, mating to the collection of replicating vectors may then be used to quickly identify a gene or genes that rescue this defect. In a related way, a vector or group of vectors (all of the LysR family of regulators, for example) may be conjugated into the strains that carry reporter genes to see which fusions are activated by which regulators.

### EXAMPLE 2

This example describes a penta-parental mating protocol for the in vivo transfer of the napA gene from S. meliloti from an entry vector to a destination vector.

As described below, the integrase reactions described in EXAMPLE 1 can be carried out in vivo, for example, by establishing an entry vector containing an insert and a destination vector in a cell expressing integrase (Int) and excisionase (Xis) (Platt et al. (2000) Plasmid 43:12-23), then selecting for transfer of the insert into the destination vector.

Using a mating that involved five parental strains, a pMK2010 derivative that contains the S. meliloti napA gene and an elimination-type destination vector were mobilized into a strain that contains Int and Xis. Recombinants were selected by transferring them into a plasmidless strain, selecting for a version of the destination vector missing the toxic ccdB gene. The selected clones have a destination vector with the napA insert and were recovered at reasonable frequency. Recovery depended on the presence of each parent and was stimulated by IPTG, which induced integrase expression. By carrying out these matings on plates en masse, the set of S. meliloti inserts from pMK2010 can be transferred to a destination vector in a few weeks.

The strains used in the penta-parental mating protocol are described in Table 1. DH5α (pNapA) is a derivative of DH5α containing the napA gene from S. meliloti inserted into the entry vector, pMK2010. DB3.1 is an E. coli strain containing a gyrase mutation that permits the propagation of vectors containing the ccdB gene (see, e.g., Bernard et al. (1993) J. Mol Biol. 234:534). DB3.1 (pRK2073) is a derivative of DB3.1 containing pRK2073, a trimethoprim-resistant plasmid capable of mobilizing plasmids that carry oriT from either RK2 or colE1 (Leong et al. (1982) J. Biol. Chem. 257:8724-30). DB3.1 (pMK2014) is a derivative of DB3.1A containing a destination vector derived from pMB419 (Barnett et al. (2000) Biotechniques 29:240-5) modified by insertion of an attR1-ccdB-attR2 cassette flanked by two yeast FRT sequences in inverted orientation. The destination vector also contains the oriT from RK2 and can be mobilized by pRK2073. DH5α pir (pXINT129) is a derivative of DH5α expressing integration factors (Platt et al. (2000) Plasmid 43:12-23). HB101R is a rifampicin-resistant derivative of HB101.

TABLE 1
Strains used in Penta-Parental Mating Protocol

| Strain | Selectable Marker(s) | Key Property |
|---|---|---|
| DH5α (pNapA) | Kan^{r} | Entry vector (attP) |
| DB3.1 (pRK2073) | Tpm^{r} | Plasmid transfer factors |
| DB3.1 (pMK2014) | Hyg^{r}, Cam^{r}, ccdB | Destination vector (attR) |
| DH5α pir (pXINT129) | Kan^{r} | Integration factors (λint) |
| HB101R | Rif^{r} | Final host |

Strains were grown in 125 ml flasks with 10 ml of LB broth+appropriate antibiotics (40 μg/ml kanamycin, 100 μg/ml trimethoprim, 50 μg/ml chloramphenicol, 100 ug/ml rifampicin) overnight in a 37° C. shaking water-bath at 240 rpm. The cultures were centrifuged at 4000×g for 5 minutes to harvest cells (6000 rpm in Sorvall SA-600 rotor), and cell pellets were resuspended in 10 ml sterile 0.85% NaCl. The centriftigation and resuspension were repeated two times. The final time, each cell pellet was resuspended in 1 ml sterile 0.85% NaCl. 20 microliters of each of the five mating partners were spotted into one of six sectors of an LB+IPTG (20 μg/ml) plate. 20 microliters of each of the five mating partners were added to a sterile (microcentrifuge) tube, mixed and 20 microliters of the mixture was spotted on the remaining sector of the LB plate. The mating plates were incubated overnight at 37° C.

The next day, an inoculating loop was used to streak some of the individual mating partners and the mating mixes onto LB plates+hygromycin (100 μg/ml) and rifampicin containing (100 μg/ml). The plates were incubated at 37° C. until colonies appeared. Only the mating mixture spot leads to colonies. These contain the destination vector, pMK2014, that now contains the napA gene.

In two sets of quantitative experiments, each with three separate matings starting with the same original cultures, the mating mixture spot was excised after overnight mating and the bacteria were resuspended in 1 ml saline and diluted for plate counts. In the first set of experiments, all five strains were mixed together and plated in the same spot. In the second set of experiments, the strain containing the entry vector was spotted on a lawn of the other four strains. In the first set of experiments, a 10-fold dilution of the suspension yielded 215, 72, and 113 colonies in the three separate matings, respectively. In the second set of experiments, a 10-fold dilution of the suspension yielded 316, 102, and 138 colonies, respectively. Therefore, this procedure yielded about 1000 exconjugants per mating. All the exconjugants that were examined contained the desired plasmid, i.e., the destination vector containing NapA.

### EXAMPLE 3

This example describes a 96-well format for the penta-parental mating protocol described in EXAMPLE 2, for the in vivo transfer of DNA sequences from an entry vector to a destination vector.

The same strains are used as described in EXAMPLE 2 and Table 1. All mating strains except the entry vector strain were grown and harvested as described in EXAMPLE 2. The four mating strains (not including the entry vector strain) were mixed together resulting in 4 mL of “Transfer mix.” Using an 8-channel micropipet, 25 microliters of “Transfer mix” were added aseptically to each well of a sterile, flat-bottom 96-well plate.

A 96-well plate was prepared with each well containing 25 microliters of a different entry vector strain. This was be done by transferring a small amount of a colony in LB medium using a toothpick or inoculating loop, then agitating the microtiter plate for several minutes on a microtiter plate shaker. Using a cooled, flame-sterilized, 96-prong replicator, 96 spots of “Transfer mix” were stamped onto a rectangular petri dish containing LB medium supplemented with 20 μg/ml IPTG. The replicator was dipped in 100% ethanol and flamed then either allowed to cool in air or set on a sterile LB agar plate. The spots were allowed to soak into the medium (this takes several minutes and works better if the plates are a little dry). A custom replicator with ˜2 mm diameter pins and fits into an alignment apparatus we used. The replicator plate was constructed as an aluminum rectangular plate into which 1¼″×⅛″ screws were inserted. The screws were then milled to flatten the ends and create a planar array.

Using the cooled, flame-sterilized, 96 prong replicator, the 96 entry vector strains were transferred directly on top of the transfer mix spots. The plates were incubated overnight at 37° C. to allow mating to occur and segregation to happen.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for moving an insert nucleic acid molecule between vectors, comprising transferring an insert nucleic acid molecule from a first vector to a second vector using site-specific recombination in vivo.

2. The method of claim 1, wherein the transferring comprises combining two or more cells, wherein each cell comprises at least one of:
(a) a first vector comprising a transfer origin and an insert nucleic acid molecule, wherein the insert nucleic acid molecule is flanked by a first recombination site and by a second recombination site;
(b) a second vector comprising a transfer origin and a first recombination site partner and a second recombination site partner;
(c) one or more recombination proteins, which mediate recombination between the first recombination site and the first recombination site partner, and between the second recombination site and the second recombination site partner; and
(d) one or more plasmid transfer factors, which mediate inter-cellular transfer of the first and second vectors;
and wherein the cells are combined under conditions effective to promote conjugation and recombination.

3. The method of claim 2, wherein the transfer comprises combining a first cell comprising the first vector comprising the insert nucleic acid molecule and a second cell comprising the second vector.

4. The method of claim 3, further comprising combining a third cell comprising one or more recombination proteins.

5. The method of claim 4, further comprising combining a fourth cell comprising one or more plasmid transfer factors.

6. The method of claim 5, further comprising combining a fifth cell that allows for selection of the second vector comprising the insert nucleic acid molecule.

7. The method of claim 1, wherein the transfer comprises combining:
(a) a first cell comprising a first vector comprising a transfer origin and an insert nucleic acid molecule, wherein the insert nucleic acid molecule is flanked by a first recombination site and by a second recombination site;
(b) a second cell comprising a second vector comprising a transfer origin and a first recombination site partner and a second recombination site partner;
(c) a third cell comprising one or more recombination proteins, which mediate recombination between the first recombination site and the first recombination site partner, and between the second recombination site and the second recombination site partner; and
(d) a fourth cell comprising one or more plasmid transfer factors, which mediate inter-cellular transfer of the first and second vectors;
wherein the cells are combined under conditions effective to promote under conditions effective to promote conjugation and recombination.

8. The method of claim 7 further comprising combining a fifth cell that allows for selection of the second vector comprising the insert nucleic acid molecule.

9. The method of claim 1, wherein the site-specific recombination comprises the integrase/att system from bacteriophage lambda.

10. The method of claim 2, wherein the first recombination site comprises an attB1 site, wherein the second recombination site comprises an attB2 site, wherein the first recombination site partner comprises an attP1 site, wherein the second recombination site partner comprises an attP2 site, and wherein the one or more recombination proteins comprise lambda integrase and Integration Host Factor.

11. The method of claim 2, wherein the first recombination site comprises an attL1 site, wherein the second recombination site comprises an attL2 site, wherein the first recombination site partner comprises an attR1 site, wherein the second recombination site partner comprises an attR2 site, and wherein the one or more recombination proteins comprise lambda integrase and Xis.

12. The method of claim 1, wherein the transfer origins in the first vector and the second vector comprise an oriT sequence from plasmid RK2.

13. The method of claim 2, wherein the one or more recombination proteins comprise lambda integrase.

14. The method of claim 13, wherein the one or more recombination proteins further comprise Integration Host Factor.

15. The method of claim 13, wherein the one or more recombination proteins further comprise Xis.

16. The method of claim 2, wherein two or more cells are bacterial cells.

17. The method of claim 16, wherein the bacterial cells are E. coli cells.

18. The method of claim 1, wherein the insert nucleic acid molecule within the second vector is operably linked to a promoter.

19. The method of claim 1, wherein the insert nucleic acid molecule within the second vector is operably linked to a reporter gene.

20. A method for analyzing the function of a genomic sequence in a prokaryotic organism using site-specific recombination in vivo, comprising:
(a) providing a first vector comprising a transfer origin and an insert nucleic acid coding molecule flanked by a first recombination site and by a second recombination site, wherein the insert nucleic acid molecule comprises a sequence from a genomic region in a first prokaryotic organism;
(b) transferring the insert nucleic acid molecule within the first vector into a second vector comprising a transfer origin and a first recombination site partner and a second recombination site partner by site-specific recombination in a second prokaryotic organism;
(c) transferring the second vector from the second prokaryotic organism into the first prokaryotic organism by conjugation; and
(d) analyzing the function of the genomic region in the first prokaryotic organism.

21. The method of claim 20, wherein the first vector and the second vector further comprise one or more selectable markers.

22. The method of claim 20, wherein step (b) comprises combining two or more cells, wherein each cell comprises at least one of:
(i) a first vector comprising a transfer origin and an insert nucleic acid molecule, wherein the insert nucleic acid molecule is flanked by a first recombination site and by a second recombination site;
(ii) a second vector comprising a transfer origin and a first recombination site partner and a second recombination site partner;
(iii) one or more recombination proteins, which mediate recombination between the first recombination site and the first recombination site partner and between the second recombination site and the second recombination site partner, respectively; and
(iv) one or more plasmid transfer factors, which mediate inter-cellular transfer of the first and second vectors;
and wherein the cells are combined under conditions effective to promote conjugation and recombination.

23. The method of claim 20, wherein the first recombination site comprises an attB1 site, wherein the second recombination site comprises an attB2 site, wherein the first recombination site partner comprises an attP1 site, wherein the second recombination site partner comprises an attP2 site, and wherein the one or more recombination proteins comprise lambda integrase and Integration Host Factor.

24. The method of claim 20, wherein the first recombination site comprises an attL1 site, wherein the second recombination site comprises an attL2 site, wherein the first recombination site partner comprises an attR1 site, wherein the second recombination site partner comprises an attR2 site, and wherein the one or more recombination proteins comprise lambda integrase and Xis.

25. The method of claim 20, wherein the second vector further comprises a third recombination site and a fourth recombination site in opposite orientations flanking the first and the second recombination sites, and wherein the third and fourth recombination sites do not substantially recombine with the first recombination site, the second recombination site, the first recombination site partner, and the second recombination site partner.

26. The method of claim 25, wherein the third and fourth recombination sites are FRT sites.

27. The method of claim 20, wherein second vector replicates in the first prokaryotic organism.

28. The method of claim 20, wherein the second vector integrates into the genome of the first prokaryotic organism.

29. A method for deleting a target region in a prokaryotic genome by site-specific recombination in vivo, comprising the steps of:
(a) introducing a first recombination site into a first genomic region by homologous recombination, wherein the first genomic region is adjacent to a first end of a target genomic region;
(b) introducing a second recombination site into a second genomic region by homologous recombination, wherein the second genomic region is adjacent to a second end of the target genomic region; and
(c) deleting the target genomic region by providing one or more recombination proteins to catalyze site-specific recombination between the first and second recombination sites.

30. The method of claim 29, wherein step (a) comprises introducing a first vector comprising a first DNA sequence and a first recombination site into a first genomic region by homologous recombination, wherein the first genomic region is adjacent to a first end of a target genomic region and the first DNA sequence is homologous to the first genomic region; and step (b) comprises introducing a second vector comprising a second DNA sequence and a second recombination site into a second genomic region by homologous recombination, wherein the second genomic region is adjacent to a second end of a target genomic region, the second DNA sequence is homologous to the second genomic region, and the second recombination site has the same orientation as the first recombination site.

31. The method of claim 29, wherein step (a) comprises homologously recombining a first vector comprising a first DNA sequence between a first and a second recombination site into a first genomic region, wherein the first genomic region is adjacent to a first end of a target genomic region, the first DNA sequence is homologous to the first genomic region, and the first and second recombination sites have different orientations; and step (b) comprises homologously recombining a second vector comprising a second DNA sequence between a third and fourth recombination site into a second genomic region, wherein the second genomic region is adjacent to a second end of the target genomic region, the second DNA sequence is homologous to the second genomic region, the third recombination site has the same orientation as the second orientation site, and the fourth recombination site has the same orientation as the first site-specific recombination site.

32. The method of claim 29, wherein the recombination sites comprise FRT sequences and the one or more recombination proteins comprise a Flp recombinase.

33. The method of claim 29, wherein the prokaryotic genome is the S. meliloti genome.

34. A DNA vector, comprising a transfer origin for conjugation and a selectable marker flanked by a first recombination site and a second recombination site.

35. The vector of claim 34, wherein the first and second recombination sites comprise att sites.

36. The vector of claim 34, wherein the selectable marker is ccdB.

37. The vector of claim 34, wherein the transfer origin comprises the oriT sequence from RK2 or ColEI.

38. The vector of claim 34 further comprising a second selectable marker.

39. The vector of claim 34, comprising the sequence provided in SEQ ID NO:1.

40. A kit, comprising one or more vectors comprising a transfer origin for conjucation and a selectable marker flanked by a first recombination site and a second recombination site, and instructions for moving one or more insert nucleic acid molecules from a first vector into a second vector using site-specific recombination in vivo.

41. The kit of claim 40, wherein the one or more vectors comprise the sequence provided in SEQ ID NO:1.

42. The kit of claim 40 further comprising cells comprising coding sequences for one or more recombination proteins.

43. The kit of claim 40 further comprising cells comprising coding sequences for one or more plasmid transfer factors.

44. The kit of claim 40 further comprising cells that allow for selection of recombinant second vectors comprising the insert nucleic acid molecule(a).
